(19) United States
(12) Patent Application Publication
(10) Pub. No.: US 2005/0112786 A1
(43) Pub. Date: May 26, 2005

(54) Method of immobilizing a substance of interest to a solid phase

(76) Inventors: Qing Wang, Hollis Hills, NY (US); Daniel M. Levine, Briarcliff Manor, NY (US); Thomas S. Parker, Brooklyn, NY (US)

Correspondence Address: FULBRIGHT & JAWORSKI, LLP, 666 FIFTH AVE, NEW YORK, NY, 10103-3198, US

(21) Appl. No.: 10/723,923

(22) Filed: Nov. 25, 2003

(51) Int. Cl. G01N 33/553

(52) U.S. Cl. 436/526

(57) ABSTRACT

Methods are described for attaching molecules to a solid phase, such as a magnetic bead. Free amine groups on the solid surface react with a molecule that contains a sulfhydryl group. The sulfhydryl group may, but need not be protected. The molecule is then reacted with a conjugate of linker and ligand. The liner has a reactive maleimide or sulfhydryl group which reacts with the molecule on the solid phase.

### FIELD OF THE INVENTION

This invention relates to methods for preparing solid phases having desired molecules, such as ligands, attached thereto.

### BACKGROUND AND PRIOR ART

The use of solid phase materials in the field of analytical chemistry is well known. Many methods for identifying, separating, or otherwise working with desired molecules rely on the use of solid phase materials to which a binding partner, or reactive partner, of a given molecule, is attached. Representative of the type of materials which can be used as solid phases are test tube or cuvette walls, glass slides, synthetic surfaces like plastics, particles, especially inert particles, and beads, such as magnetic beads. The latter are especially useful because they can be removed very easily from a solution in which they are placed.

The “ligand” that is attached to the solid phase, or reactive molecule, may be any substance that interacts with a target to react with it, to remove it from solution, etc. Various biological and biochemical molecules, including proteins, antibodies, carbohydrates, nucleic acids, and lipids may be the ligand, as may inorganic molecules, such as hormones, vitamins, antibiotics, aptamers, signalling molecules, or any other material of interest may serve as the ligand or as a reactive material.

Due to their widespread use, it is of interest to optimize the preparation and production of solid phase materials, such as those described above. The resulting materials can be used, e.g., to determine analytes of interest when the ligand is, e.g., an aptamer, a signalling molecule, an antibody or an antigen when the target molecule is an antibody or aptamer. Anytime a binding reaction of any type is of interest, one or more components of the binding reaction may be immobilized on the solid phase.

In the disclosure which follows, rapamycin is attached to magnetic beads; however, it is to be understood that the invention described herein relates generally to the attachment of any molecule of interest to any solid phase of interest, using the inventive methodologies set forth herein. These include other antibodies, including, but not being limited to, Tacrolimus (FK-506).

### BRIEF DESCRIPTION OF THE FIGURES

FIG. 1 shows one embodiment of the invention.

FIG. 2 shows a second embodiment of the invention.

### DETAILED DESCRIPTION OF PREFERRED EMBODIMENTS

To prepare the beads used in the invention, a solution of N-succinimidyl S-acetylthioacetate (“SATA” hereafter) was prepared by admixing 2.0 mg of SATA with 0.5 ml of dimethyl formamide (DMF). SATA is used herein because it contains both a protecting group, i.e., an acetate moiety, for a free sulfhydryl group, and an N-hydroxysuccinimide moiety, which is a good leaving group. This solution was combined with 4×10⁹ amine group presenting beads (M-270-amine) that had been washed thoroughly. The vial containing the beads and SATA solution were covered with argon, stoppered, and then was slowly tilted and rotated at room temperature for 30 minutes.

This resulted in attachment of SATA to the beads, via an acylation reaction with the free primary amine group on the beads. The N-hydroxysuccinimide moiety is a leaving group, as noted, supra. If the beads are not to be used immediately, the free sulfhydryl group remains protected. When the beads are to be used, they are deprotected by adding 500 μl of a deprotecting solution. This solution is prepared by combining 50 mmol of hydroxylamine hydrochloride, and 2.5 mmol of EDTA to about 80 ml of water, and solid disodium hydrogen phosphate to give a pH of 7.5, followed by addition of water to give a volume of 100 ml. This results in removal of the acetate groups, and restoration of the free sulfhydryl group.

The deprotecting solution is added to the beads, which are slowly tilted and rotated, for 2 hours at room temperature, after which the hydroxylamine hydrochloride and other by-products are removed by washing with TSMZ buffer (30 mM triethanolamine, 150 M sodium chloride, and 1 MM zinc chloride, at a pH of 7.3).

Following the deprotecting step, a solution of rapamycin-PMPI was added. Rapamycin-PMPI is a conjugate of the antibiotic and p-maleimidophenyl isocyanate. The PMPI serves as a “bridge” to link the SATA and antibiotic.

The solution was prepared by combining 2.0 mg of rapamycin-PMPI with 0.5 ml of DMF. The solution was added to the beads, and tilted and rotated at room temperature for 2 hours, or as 4° C. overnight. This procedure results in attachment of the antibiotic to the bead.

In order to determine if rapamycin was attached to the beads, a 50 μl sample (volume: 1.5 ml) of the beads was taken, thoroughly washed with DMSO (4 times, 500 μl each time), and then combined with 100 μl of DMSO and 200 μl of hydroxylamine. This solution results in removal of the antibiotic from the bead. This mixture was then incubated at 37° C., for 22 hours. Following incubation, the solution and beads were separated, via conventional means, and the solution was subjected to mass spectral analysis. If antibiotic is present in the solution, mass spectral analysis shows it, and confirms that the antibiotic is attached to beads.

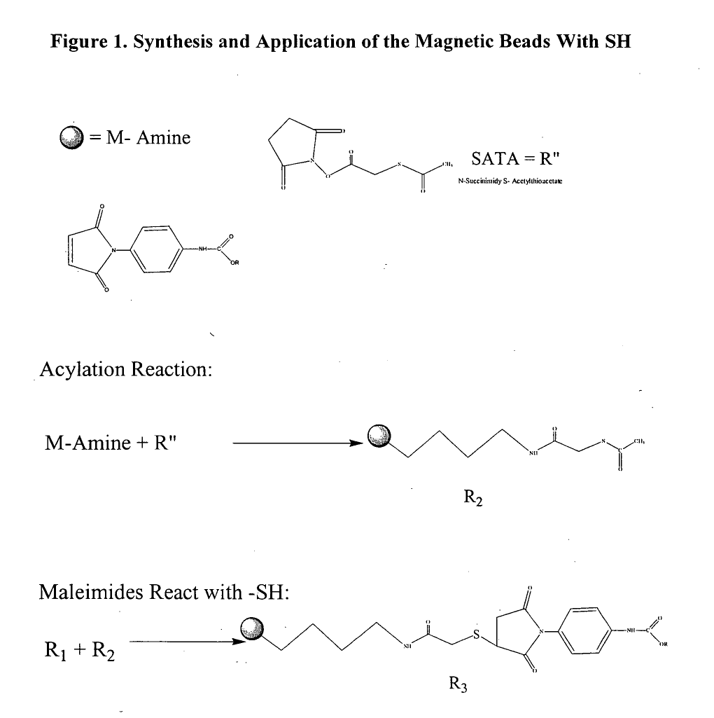

This showed that the rapamycin-PMPI had, in fact, become attached to the magnetic beads. FIG. 1 shows this procedure.

The foregoing sets forth features of the invention which related to a method for attaching a ligand to a solid phase. To elaborate, the solid phase either presents, or is treated to present, an amine group on its surface, which is free and reactive. Materials which present amine groups, as well as methods for treating solid surfaces so that they present such groups, are well known and need not be reiterated here.

The solid phase is then contacted with a molecule which is capable of reacting with the amine group, and also presents a second, reactive moiety. Such molecules are well known in the art.

For example, “SATA”, the molecule used in the examples, supra, is a member of a family of molecules in which an N-succinimidyl group and an acetyl group are joined by an alkyl chain, which may be branched or is preferably straight, and contains from 1 to 20, preferably 1 to 10, and most preferably, 1 to 5 carbon atoms. For example, when the carbon chain linking acetyl and N-succinimidyl groups contains two carbon atoms, the compound is N-succinimidyl-S-acetylthiopropionate, or “SATP”. It will be seen by the skilled artisan that additional compounds can easily be envisaged.

It will be seen from the structures of SATA and SATP, presented in the figures, that there is a reactive carbon atom. This reactive carbon atom may be replaced by an alkyl chain as defined supra, and is again, preferably from 1 to 5 carbons in length. Of course, other reactive groups may be present as well.

The second reactive group, referred to supra, must be capable of reacting with at least one of a maleimide group or a sulfhydryl group, or other groups characteristic of attachment to solid phases.

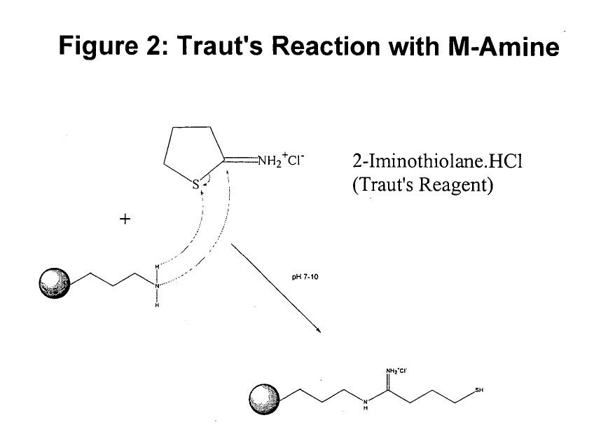

The discussion supra related to the use of molecules which contain a protective group. As discussed, supra, the acetyl moiety in SATA acts to protect the reactive sulfhydryl group, and permits the artisan to use the beads as desired. If the beads are to be used immediately, however, such protection is not necessary, and the molecule can be one such as 2-iminothiolane HCl, or “Traut's reagent” which reacts with the amine group to form a structure with a free, immediately reactive sulfhydryl group. FIG. 2 shows this reaction.

Other features of the invention will be clear to the skilled artisan and need not be elaborated upon herein.

The terms and expression which have been employed are used as terms of description and not of limitation, and there is no intention in the use of such terms and expression of excluding any equivalents of the features shown and described or portions thereof, it being recognized that various modifications are possible within the scope of the invention.

1-9. (canceled)

10. A method for attaching a ligand to a solid phase comprising:
(i) contacting an amine group on a surface of said solid phase with a molecule selected from the group consisting of 2-aminothiolane-HCl and N-succinimidyl-S-acetyl thioacetate, to provide a sulfhydryl group in said solid phase, and
(ii) reacting a conjugate of a ligand with a reactive group and a linker with the molecule attached to the surface of the solid phase in (i), wherein said linker contains a maleimide or sulfhydryl group or groups, to form a thiol linkage between said linker and said molecule; so as to attach said ligand to said solid phase.

11. The method of claim 10, wherein said solid phase is a magnetic bead.

12. The method of claim 10 wherein said ligand is an antibiotic.

13. The method of claim 12, wherein said antibiotic is rapanycin or FK-506.

14. The method of claim 10, wherein said linker is p-maleimidophenyl isocyanate.

15. The method of claim 10, comprising contacting said surface with 2-aminothiolane HCl, followed by contacting a sulfhydryl group provided by 2-aminothiolane HCl with p-maleimido phenyl isocyanate.

16. The method of claim 10, comprising contacting said surface with N-succinimidyl-S-acetylthiocyanate to provide a sulfhydryl group on said surface followed by contacting said sulfhydryl group with p-maleimido phenyl isocyanate.
